(19) United States
(12) Patent Application Publication
(10) Pub. No.: US 2025/0195888 A1
(43) Pub. Date: Jun. 19, 2025

(54) SYSTEMS, IMPLANTABLE DEVICES AND METHODS FOR VISION RELATED STIMULATION

(71) Applicant: i-Lumen Scientific, Inc., Bloomington, MN (US)

(72) Inventors: James R. Chiapetta, Delano, MN (US); Paul Rockley, Corona Del Mar, CA (US)

(21) Appl. No.: 17/782,100

(22) PCT Filed: Dec. 3, 2020
(86) PCT No.: PCT/US2020/063075

§ 371 Date: Jun. 2, 2022

Related U.S. Application Data
(60) Provisional application No. 62/942,816, filed on Dec. 3, 2019.

(51) Int. Cl. A61N 1/36; A61N 1/05; A61N 1/372; A61N 1/378

(52) U.S. Cl. CPC A61N 1/36046; A61N 1/0543; A61N 1/37264; A61N 1/3787

(57) ABSTRACT

Implantable medical devices for use in or on the eye. An implantable device may have at least one electrode thereon for issuing therapy, such as via electrical current or voltage, while implanted in or on the eye, for the treatment of one or more of macular degeneration, glaucoma, presbyopia, or other condition or disease of the eye. The implantable device may comprise a transducer for receiving power from an external device that may be worn on the patient using any of optical, inductive, mechanical or radiated energy. An example includes an implantable device for placement on the retina which has a gap or opening to place electrodes near the maculae without covering the fovea. Other examples may target other portions of the eye anatomy.

### CROSS REFERENCE TO RELATED APPLICATIONS

The present application is a national stage application of PCT Application No PCT/US2020/063075, filed Dec. 3, 2020, which claims the benefit of and priority to U.S. Provisional Patent App. No. 62/942,816, filed Dec. 3, 2019, and titled SYSTEMS, IMPLANTABLE DEVICES AND METHODS FOR VISION RELATED STIMULATION, the disclosure of which is incorporated herein by reference.

### BACKGROUND

FIG. 1 shows a prior art vision augmentation system, at a high level corresponding to the Argus II Retinal Prosthesis System from Second Sight Medical Products, Inc. The system includes an implantable subsystem 10 and an external subsystem 30. The implantable subsystem comprises a flex circuit 12 that carries an electronics case 14 and an implant coil 16, which are connected to a scleral band 18 and an electrode carrier 20 having an electrode array 22 thereon. The electrode carrier 20 is placed over the fovea of the patient's eye using a retinal tack 24, with the electrodes placed against the retina to provide electrical stimulus to neural tissues of the fovea and surrounding macula, taking the place of electrical signaling that would be generated by properly functioning light sensitive cells. The flex circuit 12 is carried on the outside of the sclera, held in place with sutures and the scleral band 18, which wraps around the sclera. The electrode carrier 20 is placed inside the eye using a pars plana incision.

The external subsystem 30 includes a camera 32 coupled to a processor 34 which couples to an external coil 36. The camera 32 is worn by the patient, for example on an eyeglasses frame, to capture an image of whatever scene or objects the patient is facing. The processor 34 converts images captured with the camera 32 and to an encoded signal that is communicated using the external coil 36 to the implant coil 16 as via an inductive link. The implant coil 16 receives the inductive signal from the external coil, receiving both power and data. The data is decoded by an application specific integrated circuit (ASIC) in the electronics case 14, and the power is received, conditioned and converted to usable electric power by electronics in the electronics case 14. The ASIC issues signals to the electrodes 22 in a pattern that is designed to be received by neural tissue in the fovea and macula. The aim is to selectively stimulate biological tissue in a spatial manner that is configured to allow the patient's visual system to recreate the images captured by the camera 32.

The system shown in FIG. 1 has been used successfully in a number of patients, however, it has several shortcomings, including complexity and size, and the difficulty of providing sufficient signal resolution to allow a patient to effectively “see.” Other proposals include the use of light responsive features, such as a photovoltaic diode, microdot or compound, in the eye that convert incident light to electrical outputs, artificially replacing the action of retinal cells that are no longer viable, as in U.S. Pat. No. 5,024,223, or adding to the action of select cells, as in U.S. Pat. Nos. 8,725,266 and 10,232,043. The aim of these existing systems and proposals is not to cause healing of the human visual system, but instead to augment cell activity or recreate images through artificial stimulus. As explained in U.S. Pat. Nos. 9,037,252 and 9,919,156, these vision replicating systems have electrodes placed on or in the fovea, where visual acuity would normally be greatest and cones are most closely concentrated.

Still further alternative proposals comprise a cortical visual prosthesis that uses a camera and a brain implant to bypass the eye and optic nerve in visually impaired patients. For such systems, significant “training” of the patient would be needed, and the final version of “sight” that the patient receives, while potentially functional to allow ambulation and some degree of object recognition, remains quite different from vision as the non-visually impaired person would understand it.

### Overview

Recreating the visual system artificially using a system such as that shown in FIG. 1 is made particularly difficult by the challenges associated with getting the brain to interpret artificially generated signals in place of those that occur naturally. An alternative approach would be to seek new methods to stall or reverse the decline in function of the biological tissue itself. Arresting or reversing such loss of function at the cellular level would present a great improvement as it would avoid the continuing challenge of interpreting the environment artificially in a manner that replicates biology. While pharmaceutical treatments are known and used for some degenerative diseases of the eye, new and alternative solutions are desired.

The present inventors have recognized, among other things, that a problem to be solved is the need for new and/or alternative systems, devices, and methods to treat, halt and/or reverse the course of eye diseases including degenerative eye diseases. In several embodiments, an implantable device is placed near target tissue in the eye to provide stimulus that can support healing or preservation of light responsive cells in the eye. In some examples, because the invention is directed to treating the tissue in the eye and/or to creating a neuro-protective or neuro-recovery function, blocking light reaching the most sensitive portions of the retina, the macula and/or fovea, may be avoided or limited.

A first illustrative and non-limiting example takes the form of an implantable system for treatment of an eye disease comprising: an implantable device having at least one electrode thereon, a control circuit, and an implantable antenna or coil for receiving an electromagnetic signal, the implantable device sized and shaped for placement in, on or adjacent to the eye such that issuance of an electric signal with the electrodes has an effect on cells of the eye; and an external device having an external antenna or coil for issuing an electromagnetic signal to the antenna or coil; wherein the control circuit of the implantable device is configured to receive power from the antenna or coil and route electrical signals to the electrodes for treatment of a disease of the eye.

A second illustrative and non-limiting example takes the form of an implantable system for treatment of an eye disease comprising: an implantable device having at least one electrode thereon, a control circuit, and an implantable transducer for receiving a power signal, the implantable device sized and shaped for placement in, on or adjacent to the eye such that issuance of an electric signal with the electrodes has an effect on cells of the eye; and an external device having an external transducer for issuing a power signal to the implantable transducer; wherein the control circuit of the implantable device is configured to receive power from the transducer and route electrical signals to the electrodes for treatment of a disease of the eye.

Additionally or alternatively, the implantable transducer and external transducer are adapted, respectively, to receive and transmit a light signal.

Additionally or alternatively, the light signal has an infrared wavelength.

Additionally or alternatively, the implantable transducer and external transducer are adapted, respectively, to receive and transmit an ultrasound signal.

Additionally or alternatively, the control circuit is configured to confirm that a signal received from the external device is not from another source.

Additionally or alternatively, the signal generated by the external device comprises both data and power, and the implantable device further comprises a demodulation circuit to extract data from the signal generated by the external device.

Additionally or alternatively, the signal generated by the external device is unrelated to any visual appearance of an object.

Additionally or alternatively, the eye disease is macular degeneration.

Additionally or alternatively, the implantable device defines an opening surrounded by the electrodes, the opening sized and shaped to avoid blocking light entering the eye from impinging on a majority of the macula.

Additionally or alternatively, the implantable device defines an opening surrounded by the electrodes, the opening sized to avoid blocking light entering the eye from impinging on the fovea centralis.

Additionally or alternatively, the opening has a diameter in the range of about 1.5 to 8 millimeters.

Additionally or alternatively, the opening has a diameter in the range of about 2.5 to about 6 millimeters.

Additionally or alternatively, the disease is glaucoma, and the electrical signals are configured for enhancing or reducing fluid movement in the eye.

Additionally or alternatively, the disease is presbyopia, and the electrical signals are configured for affecting the ciliary muscle.

Additionally or alternatively, the control circuit is programmable to define an output pattern using the electrodes as anodes and cathodes in a programmable sequence.

Additionally or alternatively, the implantable transducer comprises at least first and second transducing units that are separately activatable, and the control circuit is configured to selectively activate the electrodes depending on which transducing unit is activated.

Additionally or alternatively, the implantable transducer is adapted to receive an RF signal and convert the RF signal into electrical power.

Additionally or alternatively, the implantable transducer is an inductive coil.

Another illustrative and non-limiting example takes the form of a method of treating a patient having an eye disease comprising implanting an implantable device in a position on the retina and over or in proximity with the macula such that the fovea centralis is not covered by the implantable device, the implantable device having a plurality of electrodes thereon, wherein the implanting step is performed to place the electrodes in contact with the retina.

Additionally or alternatively, the implantable device comprises a plurality of electrodes that surround an opening, wherein the method of implantation includes placing the opening approximately over the fovea.

Additionally or alternatively, the opening has a diameter in the range of about 1.5 to 8 millimeters.

Additionally or alternatively, the opening has a diameter in the range of about 2.5 to about 6 millimeters.

Additionally or alternatively, the method may further comprise applying at least one retinal tack to hold the implantable device in place.

Additionally or alternatively, the step of applying at least one retinal tack comprises applying each off first and second retinal tacks on opposite sides of the fovea.

Additionally or alternatively, the implanting step is performed such that the implantable device does not cover the macula.

Additionally or alternatively, the method may further comprise delivering electrical outputs between the electrodes to stimulate neural activity in the maculae and/or fovea.

Additionally or alternatively, the electrical outputs are of sufficient power to cause the patient to experience phosphenes.

Additionally or alternatively, the step of implanting the implantable device is performed such that the implantable device is located entirely within the eye and does not contact the outer surface of the sclera once implanted.

Additionally or alternatively, the step of implanting the implantable device is performed such that the implantable device is placed entirely epiretinally.

Still another illustrative and non-limiting example takes the form of a method of treating a patient having glaucoma comprising implanting an implantable device in a position in the choroid, the implantable device having a plurality of electrodes thereon, wherein the implanting step is performed to place the electrodes in proximity to blood vessels that play a role in controlling fluid pressure inside the eye.

Another illustrative and non-limiting example takes the form of a method of treating a patient having glaucoma comprising implanting an implantable device in a sub-choroidal position, the implantable device having a plurality of electrodes thereon, wherein the implanting step is performed to place the electrodes in proximity to blood vessels that play a role in controlling fluid pressure inside the eye.

Another illustrative and non-limiting example takes the form of a method of treating a patient having glaucoma comprising implanting an implantable device on the outside of the sclera, the implantable device having a plurality of electrodes thereon, wherein the implanting step is performed to place the electrodes in proximity to fluid ducts in the anterior eye.

Additionally or alternatively to the three preceding illustrative and non-limiting examples, the method may further comprise activating the implantable device to issue therapy using the electrodes to reduce fluid pressure inside the eye by inhibiting fluid flow into the eye.

Additionally or alternatively to the three preceding illustrative and non-limiting examples, the method may further comprise activating the implantable device to issue therapy using the electrodes to reduce fluid pressure inside the eye by encouraging fluid flow out of the eye.

Still another illustrative and non-limiting example takes the form of a method of treating presbyopia comprising implanting an implantable device on the outside of the sclera in proximity to the ciliary muscles, the implantable device having at least one electrode thereon, and activating the implantable device to issue therapy with the electrodes to cause contraction of a ciliary muscle. Additionally or alternatively, the method may further comprise determining whether the patient's eyes are closed before activating the implantable device to issue therapy.

Yet another illustrative and non-limiting example takes the form of a method of operation in an implantable device that is implanted on or in the eye of a patient, the implantable device having at least one electrode thereon, the method comprising issuing therapy with the electrodes to cause a reduction in intraocular pressure. Additionally or alternatively, the therapy is delivered to cause choroidal blood vessels to dilate. Additionally or alternatively, the therapy is delivered to cause choroidal blood vessels to contract. Additionally or alternatively, the therapy is delivered to cause dilation of one or more of the trabeculae and/or Schlemm's canal.

Another illustrative and non-limiting example takes the form of a method of operation in an implantable device that is implanted on or in the eye of a patient, the implantable device having at least one electrode thereon, the method comprising issuing therapy with the electrodes to cause a ciliary muscle contraction.

Another illustrative and non-limiting example takes the form of a method of treating a disease of the eye comprising: with an external device, issuing a power signal in the vicinity of or directed into the eye; with an internal device located inside the eye, receiving the power signal and converting it to an electrical output; with the internal device, issuing through electrodes of the internal device, electrical voltage or current outputs to retinal cells to stimulate said retinal cells; wherein the electrical voltage or current outputs are unrelated to any image or scene. Additionally or alternatively, the internal device is located entirely within the eye and does not contact the outer surface of the sclera once implanted.

Another illustrative and non-limiting example takes the form of a method of treating a disease of the eye comprising: with an external device, issuing a power signal in the vicinity of or directed into the eye; with an internal device located inside the eye, receiving the power signal and converting it to an electrical output; with the internal device, issuing through electrodes of the internal device, electrical voltage or current outputs to retinal cells to stimulate said retinal cells; wherein the internal device is attached to the retina in the vicinity of but not covering the fovea. Additionally or alternatively, the internal device is located entirely within the eye and does not contact the outer surface of the sclera once implanted.

This overview is intended to introduce the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a prior art vision augmentation system;

FIG. 2 shows a healthy retina, macula, and fovea;

FIG. 3 shows a retina, macula and fovea in a patient having a disease of the eye;

FIG. 4 shows an illustrative implant system for treatment of an eye disease;

FIG. 5 shows implantation of an implant system for treatment of an eye disease in a side section view;

FIGS. 6-8 and 9A-9B show plan views for illustrative implantation of illustrative implant systems for treatment of an eye disease;

FIG. 10 shows in block form circuitry for an illustrative implant system for treatment of an eye disease; and

FIGS. 11 and 12 show illustrative methods of operation for treatment of an eye disease with an implantable system.

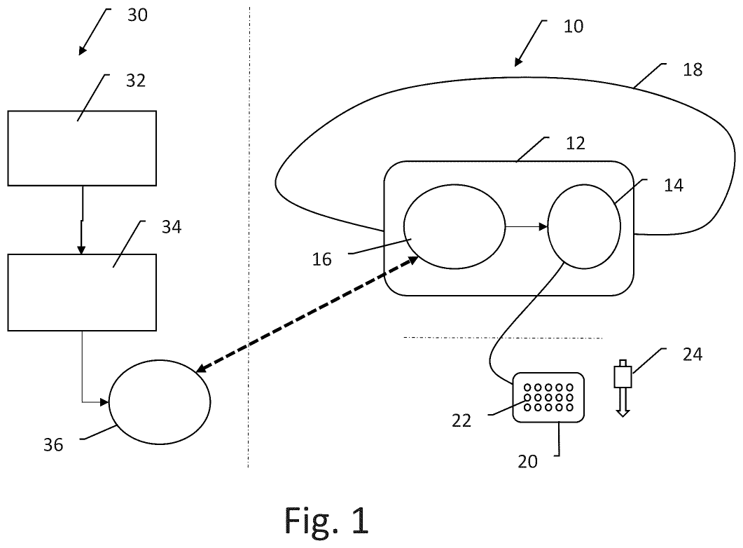

### DETAILED DESCRIPTION

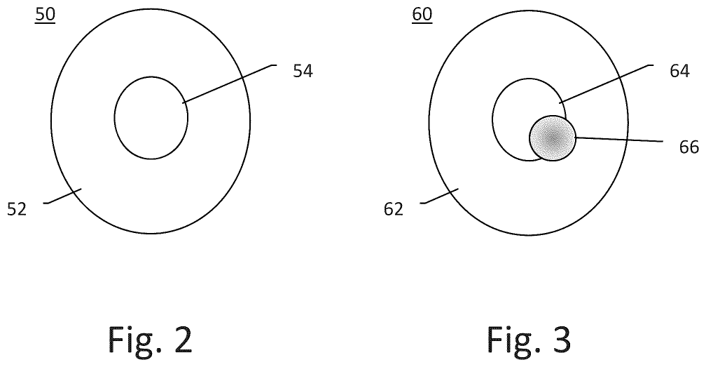

FIG. 2 shows a healthy retina, macula, and fovea centralis (fovea). The retina 50 covers the interior of the eye, and is the light sensitive layer of tissue in the eye. In the central posterior region of the retina 50 lies the macula 52, an oval-shaped area having a denser concentration of neurons than the rest of the retina 50. Images are focused by the lens of the eye on the macula, which forms the visual center of the eye. The highest density region of light sensitive cells or “cones” is the fovea 54, which is the region of the eye that is sufficiently dense in cones to facilitate 20/20 vision. The choroid contains connective tissue and blood vessels, and lies between the sclera and the retina and provide oxygen and nutrients to the retina. As used herein, the retina has an “inside” which faces the vitreous humor, and an “outside” which faces the sclera and choroid.

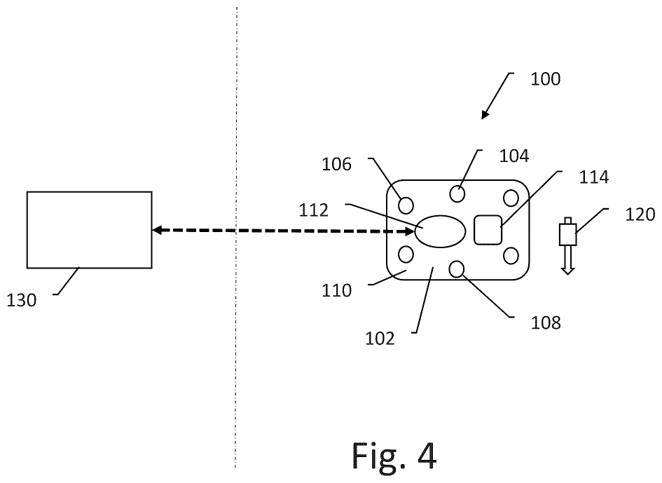

FIG. 3 shows a retina, macula and fovea in a patient having a disease of the eye, such as macular degeneration. On the retina 60, the macula 62 is shown having a diseased area 66, shown as encroaching on the fovea 64. With a condition such as macular degeneration, this diseased area 66 begins to impair the center of the field of vision of the patient by damaging cells in the fovea 64. Over time, the area 66 grows due to the degenerative nature of the disease. It has been found that electrical stimulation of the eye, such as that described in U.S. Pat. No. 7,251,528 (the '528 patent), can arrest and even reverse the progress of some diseases of the eye, such as macular degeneration. The system in the '528 patent applies energy externally to the eye such as by transcutaneously issuing electrical pulses in the area of the eyelid. Further alternatives are desirable.

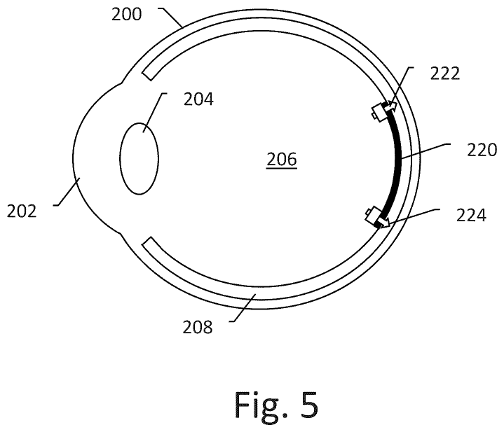

FIG. 4 shows an illustrative implant system for treatment of an eye disease. This system comprises an implant system 100 adapted for placement entirely inside of the eye. The system comprises a carrier 102 that may be held in place using a retinal tack 120. The carrier 102 may be placed at the posterior portion of the retina in an epiretinal manner (that is, on the side of the retina which faces the vitreous body). In some examples, implantation is performed without removing tissue layers that cover and protect the retina, although such tissue layers may be removed if desired in some embodiments. In another example, the carrier 102 may be placed subretinally by inserting a needle having a lumen therein through the retina to the back side thereof and injecting a fluid to create a space for receiving the carrier 102. After the fluid is injected, the carrier 102 may be inserted through the needle lumen in a rolled-up state and then unrolled once in position, for example.

The carrier 102 includes a flexible layer 110, such as a flex circuit, which carries a plurality of electrodes 104, 106, 108. The electrodes 104, 106, 108 may be coupled to a therapy generating circuit using an array of switches to allow different ones of the electrodes 104, 106, 108 to be selected as anode, cathode or off during therapy output. Any number of electrodes, from two to several dozen or even more than one hundred electrodes may be provided. The electrodes 104, 106, 108 in an example may be flat, or they may be raised relative to the carrier 102. In other examples, the electrodes 104, 106, 108 may comprise spikes that extend out from the carrier 102, which are inserted into the retina themselves, to provide greater contact to the retinal tissue or even to tissue behind the retina (if placed at an epiretinal position), if desired. While the implanted portion of the system in this example is entirely inside the sclera, in other examples the implanted portion may include parts that are inside the sclera and outside the sclera, and still other examples may include an implanted portion that is implanted on the sclera which does not pass inside the sclera.

The carrier 102 also carries a receiving element 112, which may be a transducer having any of several forms. In some examples, the receiving element 112 may be an inductive coil that can receive power and/or data signals from an external inductive coil. In other examples, the receiving element 112 may be an antenna for receiving a radio signal generated using an external antenna, such as by using an RF band frequency. Various RF and ultrasound transducers and transmission systems are known in the implantable medical device arts suitable for such designs. In some examples, the receiving element 112 may be a mechanical transducer adapted to receive power in from an ultrasound generator and convert such received power into electrical power (miniaturized ultrasound transducers are known, for example, in U.S. Pat. No. 7,610,092). In still other examples, the receiving element may include one or more photovoltaic diodes that convert received light into electrical power.

A control module 114 may be provided on the electrode carrier 102. The control module 114 may comprise a microcontroller or ASIC, depending on the complexity desired. When power is received with receiving element 112, the power may be routed to suitable circuitry to rectify and smooth the received power, such as by storing on a capacitor, to provide usable power for the control module 114. If data is received by the receiving element 112, a demodulation circuit may be provided to extract data signals, such as commands for electrode selection and characteristics of electrical outputs to be delivered via the electrodes 104, 106, 108.

Power received may, in some examples, be used as it is received such that the implant system 100 powers up after power transmission starts, and powers down once the power transmission ceases. In other examples, power may be stored, such as on a capacitor or rechargeable battery, to provide therapy at a later time, or to allow the implant system 100 to provide therapy for a longer period of time than the period during which power transmission takes place. In an example, the implant system 100 stores enough power to be able to perform a power-down sequence for an ASIC or microcontroller after therapy has been delivered. In other examples, the implant system 100 stores power more or less in an ongoing basis, using a low power mode and clock with occasional or periodic wakeups for issuing therapy pulses at predetermined times or in response to predetermined events. For example, the implant system 100 may include a light sensitive element that determines when the patient's eyes are closed, and therapy delivery may occur when the eyes are closed. This could provide a benefit if, for example, the therapy delivered causes elicitation of phosphenes that may be undesirable to the patient while engaged in an activity such as driving. The implant system 100 may instead deliver therapy at a predetermined time of day. Some examples may omit power transmission by including a non-rechargeable or “primary cell” battery in the implant system 100, and may use a communication system to receive therapy commands indicating when and how therapy is to be delivered.

An external device 130 is shown as well. The external device 130 may be a wearable item, such as a patch, frame, eyepiece, headband, etc. that is provided in close proximity to the eye. For example, the external device may be held, worn, or attached to the temple, to an eyelid, or elsewhere, allowing issued signals, whether inductive, mechanical (ultrasound), or radio waves, to be efficiently coupled to the tissue. In some examples that use light as a transmitting medium for power and/or data, the external device 130 may be worn in front of the eye, such as on an eyepiece or on a glasses frame, to allow light to be directed into the eye, thereby illuminating the retina where the implant 100 is placed. When light is used, in some examples, the issued light signal carrying power and/or data is within the visible spectrum (approximately 380 to 700 nm). In other examples, the issued light signal carrying power and/or data is outside of the visible spectrum (such as using an infrared or ultraviolet laser spectra, or using wavelengths still farther from the visible spectrum).

In one example, the wearable element 130 comprises an inductive coil that can be held on the temple or forehead, or over an eyelid, and the implant 100 receiver 112 is an inductive coil. The inductive signal may be generated at any suitable frequency, such as, and without limitation, a frequency in the range of 25 to 250 kHz, or higher or lower. The implant device 100 receiver is also an inductive coil and is tuned to about the same frequency as the external element 130. Power transmission is achieved by the inductive coupling of the coils, and any suitable method for data transmission (if included) can be used to also convey data, including, for example, frequency shift keying (in which the center frequency of transmission is varied) or on-off keying (in which output power is modulated). As noted, other examples may use mechanical (ultrasound), light-based, or radio waves instead.

Of note, in the system provided, several distinctions are already apparent over prior art systems that seek to replicate vision. For example, the signals generated by the external device 130 and received by the implant 100 may be unrelated to anything external to the patient; that is, a scene that the patient is looking at is not being replicated, and no attempt is made in some embodiments to use the transmitted power to cause the patient to see any particular object or scene. Thus electrical stimulus is delivered in some embodiments without imparting a pattern of stimulation that is related to a scene, object, objects, or image. The complex manipulations and data transmission requirements of vision replication systems can be omitted. For embodiments that do not attempt to recreate an image for the patient, the quantity of electrodes can be reduced greatly from artificial vision systems, and the size of electrodes can be larger than with high resolution vision replacement systems. The external device 130 may omit a camera, and may omit processing capacity for receiving and encoding data from an observed scene. The transmission to the implant 100 may omit data encoded by the external device 130 from an observed scene or object(s). However, the present invention is not necessarily limited to the omission of vision replication, and, in alternative embodiments, such vision-recreating capability may also be included.

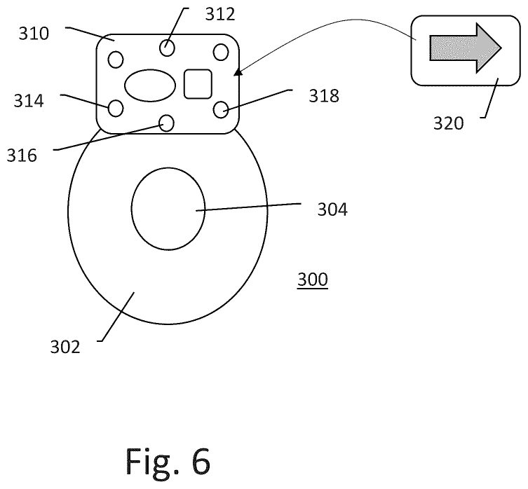

FIG. 5 shows an example of implantation of an implant system for treatment of an eye disease in a side section view. In this example, the eye 200 is shown with the anterior portion thereof at 202, with the lens at 204 (other structures being left out of the diagram for simplicity). Inside the eye is the vitreous body 206 and retina 208. An implant 220 is shown placed at the rear portion of the retina 208, in the region where the maculae and fovea are. One or two (or more) retinal tacks 222, 224 may be used to secure the implant 220 in a desired position. In some examples, the material of the implant 220 may be selected to be “clear” in the sense that it will not block reception of incident light at the maculae or fovea; in such an example the implant 220 may be placed directly over the fovea, if desired. In other examples, as shown further below, the implant 220 is placed so it is near but does not cover at least the fovea. In still other examples, the implant 220 defines an opening that goes over at least the fovea and/or the maculae. Rather than a solid flex circuit, the implant 220 may rely on an insulated wire mesh having openings therethrough allowing incident light to pass through to the maculae.

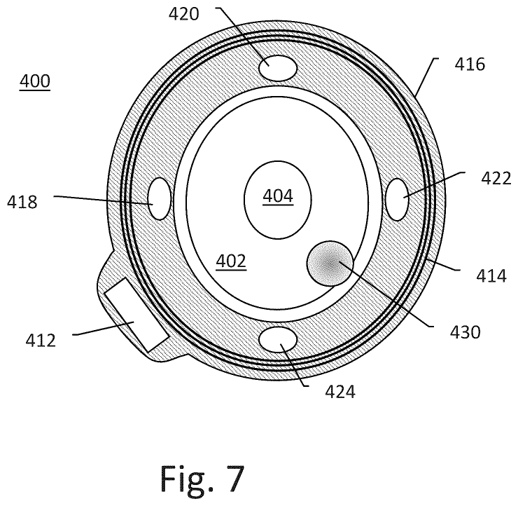

FIGS. 6-9 show a plan views for illustrative implantation of illustrative implant systems for treatment of an eye disease. In FIG. 6, the retina is shown at 300 with the macula at 302 and fovea at 304. An implant 310 is placed near the fovea 304 but does not cover the fovea 304 itself. In this example the implant 310 is shown covering a portion of the macula 302 but it may, if desired, be placed farther from the fovea 304 so as not to cover the macula 302 at all. The implant 310 carries several electrodes 312, 314, 316, 318, which may be selectively used as anodes and/or cathodes for therapy output purposes. It has been found that electrical stimulation in the vicinity of damaged macula can activate the macula to heal itself, encouraging survival and/or recovery of the cells in the area where stimulation takes place. The immune response of the tissue in the area may further extend the survival and/or recovery of the cells.

The implant 310 may include a marker such as that shown in an enlarged view at 320 to indicate how the implant has been oriented relative to the eye. Such a marker 320 may aid in programming the use of the electrodes. For example, when implanted as shown, the electrodes at 314, 316, 318 may be used as “active” electrodes to deliver either cathodal or anodal stimulation, with electrode 312 used as a return electrode. In other examples, stimulation may be provided between the electrodes closest to the macula 302, such as by using electrodes 314, 316 and 318 only, and omitting the use of electrodes that are farther from the macula 302. In this way the surgeon does not have to be concerned with the precise orientation of the implant 310 relative to the macula 302 and fovea 304, instead focusing on where, relative to the fovea 304 and/or macula 302, the implant is placed. Once the device is placed, the directional orientation of the implant 310 may be determined using the marker 320, and therapy patterns can then be defined using electrodes as needed.

In terms of programming, therapy may be delivered by first determining an activation threshold for the individual electrodes 312, 314, 316, 318, such as by issuing output pulses and modifying one or more of amplitude, pulse width and/or frequency until phosphenes are observed by the patient. This process can be used to determine a phosphene threshold for the patient. Therapy can be delivered above the phosphene threshold in some examples, such as by setting the amplitude at some voltage or current level above the phosphene threshold using a fixed amount or percentage of the phosphene threshold. Therapy may instead be delivered at the phosphene threshold. In still other examples, therapy can be delivered below the phosphene threshold by setting the amplitude at some voltage or current level below the phosphene threshold using a fixed amount or percentage of the phosphene threshold. In other examples, after determining a phosphene threshold at a given pulse width, the pulse width may be manipulated to be “above” threshold by extending the pulse width, or “below” threshold by shortening the pulse width. In other examples, after determining a phosphene threshold at a given frequency (which may also be called the pulse repetition rate), the frequency may be manipulated to be “above” threshold by increasing the frequency, or “below” threshold by reducing frequency. A phosphene threshold can be determined for each electrode or electrode combination, if desired. In some examples, a single combination of electrodes may be used to deliver therapy. In other examples, spatial diversity of therapy, allowing targeting of various tissues, can be provided by using plural combinations of electrodes to deliver therapy.

Therapy itself may be monophasic or biphasic, and may be current or voltage controlled, and may include the use of sinusoidal or pulse based outputs, such as by delivering square waves. Other pulse shapes may be used, such as triangular, exponentially increasing or decaying, etc. For example, outputs may be issued using charged capacitors, to yield an exponentially decaying voltage, which can be truncated if desired at any selected level (10%, 50%, 60%, etc.). Outputs can be current or voltage controlled to provide a flat or square output. In another example, a sinusoidal output is generated by driving an oscillator at a selected frequency or frequencies. In other examples, a modulated waveform can be delivered as, for example, by delivering relatively higher frequency square wave or sinusoid in relatively lower frequency bursts.

Additionally or alternatively, waveform parameters and wave shapes may be generally as suggested in any of US PG Pat. Pub. No. 2020-0324114, titled SYSTEMS AND INTERFACES FOR OCULAR THERAPY, US PG Pat. Pub. No. 2020-0101290 titled SYSTEMS AND METHODS FOR CONTROLLING ELECTRICAL MODULATION FOR VISION THERAPY, US PG Pat. Pub. No. 2020-0171307, titled HEAD WORN APPARATUSES FOR VISION THERAPY, U.S. patent application Ser. No. 16/900,115, filed Jun. 12, 2020, titled WEARABLE MEDICAL DEVICE, PCT Pat. App. No. PCT/US2020/039776, filed Jun. 26, 2020, titled SYSTEMS AND INTERFACES FOR OCULAR THERAPY, and/or PCT Pat. App. No. PCT/US2020/041166, filed Jul. 8, 2020, titled OCULAR THERAPY MODES AND SYSTEMS, the disclosures of which are incorporated herein by reference, in some instances with somewhat lower amplitude/power levels as these other applications generally suggest wearable devices.

While some embodiments herein are described in terms appropriate for an electrical output, other therapy modalities such as by generating light signals, vibratory signals, or magnetic signals, may be used instead.

FIG. 6 shows the implant 310 placed on the epiretinal surface. In another example, the implant may be placed in a choroidal location or subretinal, between the retina and the sclera. Such placement may be achieved, in an illustrative and non-limiting example, by first inserting a needle to a desired position, with a lumen opening from the needle behind the retina, and injecting fluid to create a space for placing the implant 310. As noted, the implant 310 may be rolled or folded and passed through the interior of the needle lumen into position, where it can unroll or unfold.

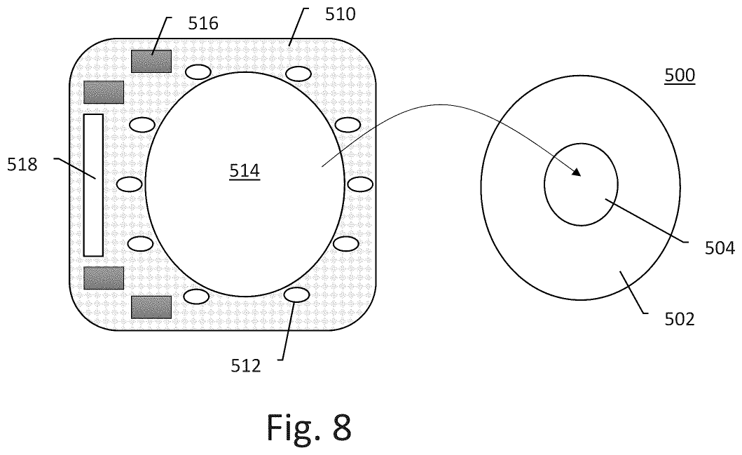

FIG. 7 shows another example. The retina is shown at 400 with the macula at 402 and fovea at 404. The implant 410 in this example is generally annular, sized to surround a target tissue by having an opening with a desired diameter. The opening can be any desired shape, such as circular, oval, polygonal, etc. In an example, the opening has a diameter in the range of about 1.5 to 8 mm. In another example, the opening has a diameter in the range of about 2.5 mm to 6 mm diameter. In another example, the opening has a diameter in the range of about 5 to 6 mm, to surround the macula. In an example, the opening has a diameter of about 5.5 mm, for example, to surround the macula 402. In another example, the opening is about 2.5 mm in diameter, to surround the fovea 404 while covering a portion of the macula 402 away from the fovea 404. A range of implant sizes may be provided. For example, a person having more advanced disease of the eye may have a smaller diameter implant placed to focus therapy at the fovea, with the understanding that portions of the maculae may be sufficiently diseased as to be possibly unrecoverable, while a person having a less advanced disease of the eye may receive an implant with a greater opening diameter. The ability to selectively target a diseased tissue area, while avoiding blocking the fovea from incident light may, in some examples, be a feature of the system.

The example shown includes a carrier 416 that carries electronics at 412, and has an inductive loop 414 that is integrated into the annular region of the implant 410, allowing a relatively larger loop to be used to potentially aid in the receipt of power (larger area being indicative of greater magnetic field capability). In another example, the feature shown at 414 may be an RF antenna; taking advantage of the full circumference of the annulus of the implant 410 may facilitate use of a longer antenna which will improve efficiency for power purposes. The carrier 416 may be, for example, a flex circuit having one or more layers of conductive connectors (not shown) to couple the electrodes, 418, 420, 422, 424 to the electronics 412 and inductive loop 414. The shape may be, for example, stamped, cut, laser cut, etc. in any suitable fashion.

In some examples, the implant may be symmetric, such as being circular or oval. In the example shown, the implant is designed with asymmetry that can be assessed after implantation, using for example, a retinal imaging procedure. The asymmetry shown, illustratively and without limitation, includes a tab extending out from the outer periphery of the carrier 416 that carries the electronics module 412. Such a shape provides an indication of the orientation of the device relative to features of the retina including the fovea and/or macula, which may aid in configuring output therapy and electrode selection to target diseased tissue, as further discussed below. In other examples, shape asymmetry may be omitted and instead the device may have one or more markers that will be apparent or visible on a retinal image, or using other imaging modality, or simply by viewing with an ophthalmoscope.

An inert, and if desired, dielectric, coating may be applied, such as a silicon or biocompatible, flexible polymer on the surfaces of the implant 410, with such coating removed or omitted in the vicinity of the electrodes 418, 420, 422, 424, if desired. The electrodes may be made of and/or incorporate layers or coatings formed of various suitable materials, such as titanium, platinum, palladium, iridium, gold, silver, niobium, titanium nitride, iridium oxide, ruthenium, ruthenium oxide, rhodium, carbon nanotubes, porous carbon, and/or metal alloys or metal layers, etc., though the present invention is not limited to any particular electrode material. In some examples, in addition to the electrodes 418, 420, 422, 424 that are shown, an additional electrode may be provided on the surface that faces away from the retina, for use as an indifferent or “remote” electrode during therapy delivery.

Four electrodes 418, 420, 422, 424 are shown in this example; more or fewer electrodes may be used if desired. Several therapy output patterns can be used to provide therapy to “paint” the region within the annulus or to target a particular area for extra stimulation relative to other areas. For example, a therapy pattern may be:

| Step | Anode(s) | Cathode(s) | Off |
|---|---|---|---|
| Step 1 | 418 | 422 | 420, 424 |
| Step 2 | 420 | 424 | 418, 422 |
| Step 3 | 422 | 418 | 420, 424 |
| Step 4 | 424 | 420 | 418, 422 |

Other patterns may be used instead. In some examples, a therapy output may use each step for a single, or small number, of pulses before moving to the next step. In other examples, therapy of Step 1 may be performed for a desired period of time, such as (and without limiting the invention) 1 second to 15 minutes, before moving to the next Step.

For this and other examples herein, illustrative pulse repetition rates or frequencies may be in the range of about 1 to 100,000 Hertz. In some examples, a frequency in the range of about 20 to 10,000 Hertz is used, or about 25 Hertz. Illustrative pulse widths may range from about one microsecond to about one hundred milliseconds. In some examples, pulse width may be in the range of about 10 microseconds to about 40 milliseconds. Amplitudes of therapy output may be, for example, in the range of microamps to milliamps, for example, in the range of about 1 microamp to about 10 milliamps, or more or less. Therapy amplitude may instead be measured or reported in terms of voltage, ranging between approximately 1 millivolt to about 100 millivolts, or more or less.

It should be recognized that the action potential for a neural cells can typically be achieved, in the absence of other stimulus, with a 15 millivolt perturbation from the resting action potential. That is, for example, a resting transmembrane potential of −70 millivolts is typical, and reaching a transmembrane potential of about-55 millivolts will cross the action potential threshold and trigger depolarization or “firing” of the neural cell. Such firing of the neural cells may cause the patient to experience a phosphene, which is often described as a lightening or a flash of light. In the eye, incident light may play a role in reducing the required applied voltage to cause such firing; in diseased tissue, however, the cells do not respond to light in the normal manner of healthy tissue. Ultimately, the exact voltage required may vary depending on the patient's disease type, disease state, incident light, and/or physical location of target cells relative to the system electrodes. It may be sufficient in some patients to “nudge” the cellular baseline to cause cell firing without needing to artificially span the entire delta from baseline that would be necessary to trigger neuron firing. It may also be unnecessary to achieve neural firing in order to cause tissue to begin healing itself and, for example, local immune response may be triggered without neural firing. In addition, the stimulus may not cause an action potential but could cause modulation of any cellular activity such as ion channel operation, upregulation of protein synthesis, energizing mitochondria or other modulation (upregulate, downregulate or hold steady) of intracellular, extracellular or cell to cell function.

Another pattern may be, for example:

| Step | Anode(s) | Cathode(s) |
|---|---|---|
| Step 1 | 418 | 420, 422, 424 |
| Step 2 | 420 | 418, 422, 424 |
| Step 3 | 422 | 418, 420, 424 |
| Step 4 | 424 | 418, 420, 422 |

The polarity may be reversed, if desired. If desired, spatial knowledge of where diseased tissue is located may be used to define a therapy protocol, such as by observing that a diseased region is located at 430, and using a pattern that targets the diseased region to have currents pass therethrough. For example, assuming a diseased region at 430, the following pattern may be used:

| Step | Anode(s) | Cathode(s) | Off |
|---|---|---|---|
| Step 1 | 422 | 424 | 418, 420 |
| Step 2 | 424 | 422 | 418, 420 |
| Step 3 | 422, 424 | 418, 420 | n/a |
| Step 4 | 418, 420 | 422, 424 | n/a |

To avoid electrode interface issues, including in particular possible oxidation or other degradation, charge delivered though each electrode may be balanced out to zero. This may be achieved by using a biphasic waveform, if desired, or by using monophasic waveforms and tracking electrode usage to ensure each electrode is used equally as anode and cathode.

In another example, the aperture or opening in the implant 410 may be sized and shaped to surround the diseased tissue area 430, rather than the maculae 402 and/or fovea 404. Thus a smaller implant or a larger implant may be provided. The implant and electrodes may be used as previously described, but in this example the target therapy will be inside the aperture of the implant. Thus cross-aperture electrode pairings, for example, may be relatively more usable as those will direct current across the diseased tissue itself.

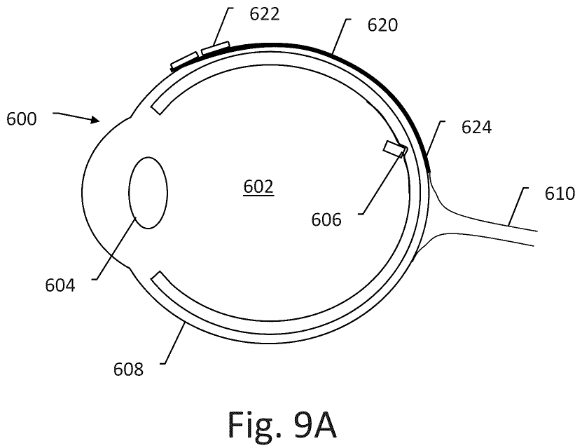

FIG. 8 shows another illustrative implantable device at 510. The device can be implanted on the retina 500, generally near the maculae 502 and fovea 504, as indicated by the line and arrow. The device 510 may take the form of a flex circuit or otherwise flexible device having electrical connections therein or thereon. A plurality of electrodes 512 are provided around an opening 514 which is sized to surround the macula 502. In other examples, the opening 514 may be sized to surround the fovea 504, if desired. For placement purposes, the opening 514 may be centered over a targeted diseased tissue if desired, or it may instead be centered as shown in FIGS. 7-8 such that the electrodes generally surround but do not cover the fovea 504 and/or macula 502. The device 510 also carries an electronics module 518 which may comprise an ASIC, logic circuitry, etc., as described relative to preceding examples and also as shown further below. In this example, power receiving elements are shown as photovoltaic devices 516.

One or several photovoltaic devices 516 may be provided, as shown. The photovoltaic devices 516 may each be one or more photodiodes, for example. Any suitable chemistry may be used, including for example, silicon, germanium and other known photodiodes, though consideration should be made to ensure adequate sealing to prevent escape of toxins, for example if a mercury cadmium telluride chemistry is used. Filtering and other features may be used to limit the bandwidth of incident light to which the photovoltaic devices 516 are sensitive to avoid inadvertent activation and/or to enable selectivity as discussed below.

In some examples the photovoltaic devices 516 may each be of the same or very similar design, capable of converting incident light, such as an infrared wavelength, into electrical energy output as a voltage which can in turn be used to power the circuitry of the electronics module 518 and to deliver electrical stimulus at the electrodes 512. In some examples, the spatial orientation of the photovoltaic devices may be used to selectively power one of the photovoltaic devices without powering other of the photovoltaic devices, an approach that may allow data to be communicated via selective activation. In another example, one or more of the photovoltaic devices is operable at a different wavelength than others of the photovoltaic devices 516. By varying the incident light wavelength, such as by using two different wavelength sources and alternating between the two, both power and data may be communicated. In one example, a system may have a number of electrodes 512 equal to the number of photovoltaic devices 516, such that selective activation of external light sources can selectively activate a single electrode or electrode pair, allowing the device to omit any electronics module 518; a further implementation may include a simple switch matrix to allow, for example, three photovoltaic devices to be activated selectively in combinations that in turn selectively activate switches in the switch matrix to facilitate the use of more electrodes than photovoltaic devices for therapy purposes. Communication may also be achieved by the use of frequency shift or on-off keying, or amplitude or frequency modulation, as desired.

In another example, again referring to FIG. 8, the power receiving elements may be mechanical transducers, rather than photovoltaic devices 516. For example, one or more mechanical transducers may be provided that are capable of receiving mechanical energy such as from an ultrasound transducer, and converting the received mechanical energy into electrical energy. Individual ones of the transducers may be tuned to different frequencies, and the incident energy varied to activate different ones of the transducers to facilitate both power and data transmission. In another example, outputs of one or more transducers may be monitored to allow demodulation of encoded data in the power signal, which may be encoded through frequency shift or on-off keying, or amplitude or frequency modulation.

In still other examples, more than one transmission receiving device may be provided on the implant device 510 using plural modalities. For example, an RF circuit (such as a low power Bluetooth circuit or a Medradio band circuit) may be used for control and communication purposes, while a mechanical, inductive, or light-based element is used for power reception. In an example, the implant 510 may include an energy harvesting device and circuit which charges a capacitor or rechargeable battery throughout the day and night, for example, receiving and storing energy in response to eye movements, and a control circuit may periodically use the stored energy to provide a therapy session. In another example, the implant 510 may include a photovoltaic device that receives incident light as the patient goes about ordinary activities and stores such energy in a capacitor or rechargeable battery, which can then be periodically discharged. In some such examples, power is stored over a relatively long period of time and discharged in a relatively short period of time, for example, by charging during a 24 hour period and discharging in a therapy session of less than an hour.

FIGS. 5-8 each show an implantation position that is generally directed to the treatment of a macular condition. Additional embodiments may be designed to treat other diseases that affect other tissue, such as glaucoma, which is thought to generally result from a failure to maintain correct fluid pressure in portions of the eye, and presbyopia, which is a lens condition that arises, typically with age, as the lens loses elasticity. In an example of glaucoma, the implantation position can be within the ciliary body or at a location directing energy to the ciliary body. Such energy may be used to cause dilation in the trabeculae meshwork, or in a one or more targeted ducts or canals in the area such as Schlemm's canal.

In an example, electrodes may be placed in the choroid or in a subchoroidal position (between the sclera and choroid) to stimulate fluid drainage via the venous network, which may be useful to reduce intraocular pressure associated with glaucoma. This may allow an alternative glaucoma treatment without impacting anatomy used in other treatments (such as those using shunts placed in Schlemm's canal). Positioning and stimulus may be directed to any suitable vascular channel as well to increase blood flow or to decrease blood flow, as conditions may require, to address other conditions. For example, increased blood flow may be useful to provide additional oxygenation that can help some tissues repair themselves. Decreased blood flow may be used to reduce fluid infusion in the area via the bloodstream, potentially reducing ocular pressure. An electrical stimulation device may also be placed in, or outside of but near, Schlemm's canal to help drain fluid and reduce glaucoma-related intraocular pressure.

An implanted device may be placed near the anterior retina, such as near the ciliary muscle, to provide electrical stimulus to the ciliary muscle. For such an implant, electrical stimulus may be provided to cause muscle contraction that can strengthen the ciliary muscles and/or limit progressive stiffening of the lens. In an example, an implanted device may be adapted to receive and store power while the patient is awake, and to then sense, by use of an optical sensor, the lack of incident light when the patient closes his or her eyes for sleep, when the illustrative device issues stimulus to the ciliary muscle, strengthening the muscle without affecting waking vision. Rather than implantation of such a system inside the eye and inside the retina, the system may be placed in an anterior eye position in the choroid. In still another example, the device may be placed on the outside of the sclera using stitches or using a scleral band, with at least an electrode carrier thereof extending to the anterior aspect of the eye. In still another example, the system may be implanted, either entirely or with an electrode carrying portion thereof, in the conjunctiva at a position that would be close to the ciliary muscle. In still another example, rather than or in addition to providing ciliary muscle stimulus during sleep to maintain muscle strength, the system may enable ciliary muscle stimulation at user command, for example to add to muscle contraction when reading, or to relax muscle contraction when performing a distance activity such as walking. In addition to responding to a user input, the device may sense myopotentials created by the flexing of the ciliary muscle, and may apply additional stimulus in response to sensed muscle activation, augmenting the contraction when the user's eye is attempting to focus.

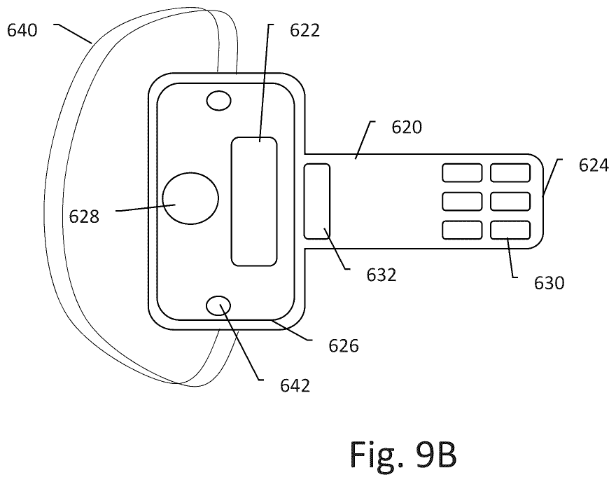

FIGS. 9A-9B show another example. Starting with FIG. 9A, the patient's eye is shown in section view at 600 having the vitreous at 602 behind the lens 604 (various structures are omitted for simplicity). Arrow 606 indicates, generally, where the retina and fovea would be located, near where the sclera 608 intersects with the optic nerve 610. The implanted device 620 in this example is implanted on the outside of the sclera 608. In the example, an electronics module is shown at 622, with a portion of the device 620 extending posteriorly to the region of the fovea. Preferably the posterior portion 624 of the device 620 stops short of reaching the location of the optic nerve 610 to avoid creating any irritation or damage thereto.

FIG. 9B shows additional details of implant 620. An electronics module is shown at 622 on an anterior portion of the implant 620, with one or several electrodes 630 on the posterior portion 624. If desired more or fewer electrodes may be used. In the example, a return electrode is illustrated at 632 having, optionally, a larger area than other electrodes 630. The implant 620 also carries a capacitor or rechargeable battery 628 which is adapted to receive and store power from an antenna or coil 626. In the example, the antenna or coil 626 is provided so as to take advantage of the larger area available in the anterior portion of the device 620.

The implanted device 620 may be held in place with one or more sutures, which may be applied using suture holes 642, if desired. Optionally a scleral band 640 may be used to hold the device 620 in place. In some examples, the entire device 620 may be designed to assume a curved shape when implanted so as to hold the posterior portion 624 in contact with the posterior portion of the sclera, which may allow a posterior stitch or anchor to be omitted. In other example, the posterior portion 624 may include a surface feature, such as a protrusion, to hold it in place on the posterior of the sclera once implanted. The various features of power and data reception, electronics, etc. described for preceding example, and also which follow, apply equally to the example in FIGS. 9A-9B.

An implantable microstimulator may be placed on or around the eye, such as in a retrobulbar position, to deliver voltages or currents to a desired portion of the eye, such as the retina or fovea, optic nerve, choroid, lens, ciliary muscle, anterior chamber, aqueous humor, etc. For example, a microstimulator may be as disclosed in any of U.S. Pat. Nos. 6,735,474, 5,193,539, 5,193,540, 5,312,439, 8,612,002, 8,886,339, and/or 10,314,501. In other examples a microstimulator may include circuitry and a battery (with or without recharging capability) similar to those known for leadless pacemakers, such as in U.S. Pat. Nos. 7,937,148, 8,010,209, 9,925,386, 9,808,633, 8,478,408, and/or 8,923,963. Implantation may be by needle injection or by the use of an implantation tool that allows placement inside the eye or in the eye socket, next to the eye, such as in the soft tissue of the area.

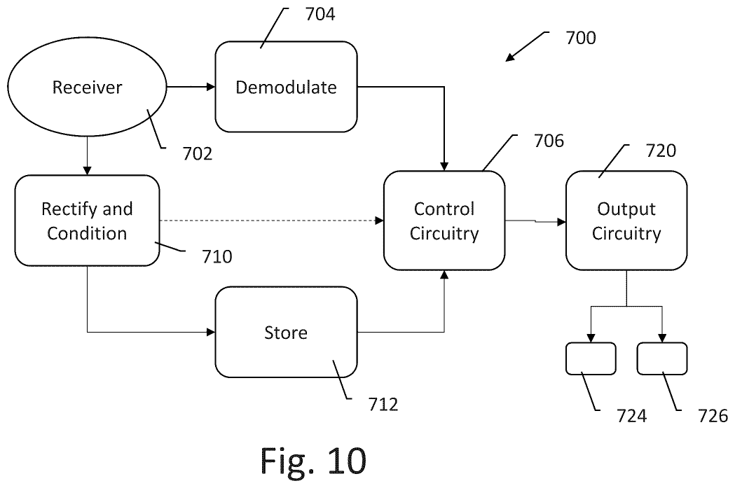

FIG. 10 shows in block form circuitry for an illustrative implant system for treatment of an eye disease. The device is shown as comprising a receiver at 702, which may take the form of a transducer for receiving mechanical energy, such as ultrasound vibrational energy, an antenna, a coil, or a photovoltaic device. The receiver 702, when energized with appropriate power, provides one or more outputs that may be handled by a demodulation circuit 704 that transfers data to a control circuitry 706 such as an ASIC, as well as an output that is handled by rectification and conditioning block 710 which powers a storage block 712, which may be for example a rechargeable battery or a capacitor. In some examples, the storage block 712 may be omitted, and the power input, once rectified and conditioned (if needed), is directly provided to the control circuitry 706 where the incoming power is used to provide control functionality as well as powering a therapy output via the output circuitry 720, which in turn selectively activates a plurality of electrodes 724, 726. Though not shown, the demodulation circuitry 704 may receive power for its tasks directly from rectify and condition block 710, or from power storage block 712.

In an illustrative example, the control circuitry 706 comprises an ASIC, as noted above. The ASIC may include, for example, a plurality of inputs, such as a set of high input impedance operational amplifiers including one or more for receiving data signals from the demodulation block 704, with a clock provided on the ASIC or in association with the ASIC to synchronize operations throughout the electronics. Control logic can be provided such as a programmable gate array or other logic subcircuits to execute operations of the system, including those which may be stored in memory associated with the control circuitry. The control circuit 706 may comprise data storage registers that may be written to with device status, history or settings; such registers may be read out in response to an interrogation signal received via the demodulation block 704 to provide device status, history or settings to be communicated to an external device. The control circuitry, with or without an ASIC, may comprise a microcontroller, if desired, or a state machine. A microcontroller, if provided, may be used to perform many of the tasks just noted. In some examples, the circuitry used may be or may resemble that which is used or has been described for use in other implantable systems such as implantable microstimulator systems or leadless pacemakers, which have volumes of less than 1 cubic centimeter in some instances.

The output circuitry 720 may include digital to analog conversion circuitry, current mirrors, voltage outputs, amplifiers, and other circuitry suitable for issuing outputs to the electrodes 724, 726. In some examples the output circuitry 720 comprises several current sources and current sinks, or several positive or negative voltage sources, which are coupled by a set of switches, such as a multiplexor or switch array, to selectable combinations of the electrodes. In other examples a dedicated power source for each electrode may be provided, omitting the switches. If desired or needed, step-up and step-down circuits may be provided to convert a power supply voltage to a higher or lower level as appropriate for therapy purposes. The output circuitry may include DC blocking or isolation capacitors associated with each of the electrodes, if desired. The output circuitry 720 may also include sensing subcircuits to allow the control circuitry 706 to determine, for example, output impedance encountered during therapy or to sense other features, such as the amount of light present in the vicinity of the device or inside the eye, which as noted above may be used to determine whether and when to issue therapy.

In some examples, the control circuitry can be more limited and may simply pass through signals to the output circuitry from the receiver. For example, in a device having multiple power receiving transducers (such as 3 such transducers) that are each separately activatable by an external device, the control and output circuitry may be simplified such that each transducer is used to power one electrode combination. An even simpler device may have a single transducer to receive power and may be used to output a single therapy. For example, a receiving coil, antenna or transducer may generate an electrical signal that is stored on a capacitor until a threshold voltage is reached (which may be determined by using something as simple as a diode), at which point power on the capacitor is dumped into an output circuit, generating a stimulus delivery. Rather than one capacitor, two may be provided in parallel for charging purposes, with the two capacitors discharged sequentially but in opposing direction, to provide a biphasic output, resulting in a charge neutral electrode interface.

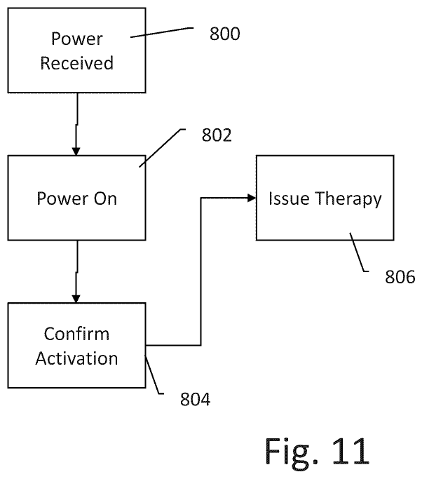

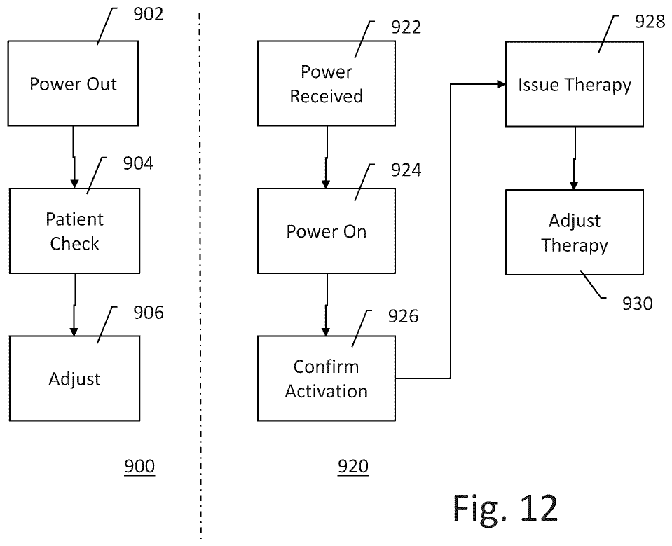

FIGS. 11 and 12 show illustrative methods of operation for treatment of an eye disease with an implantable system. FIG. 11 shows a first set of features. Here, power is received at block 800. Once a sufficient amount of power has been received, the features of an implant system are powered on as indicated at 802, which may include the performance of suitable initialization routines for any processing circuitry and/or ASIC in the device. Once power is stabilized, the implant next confirms that the incident power is in fact for use to activate therapy. For example, it may be that certain of the power modalities described above could receive power from a system that is not part of a therapy system. This may occur, for example, due to incident noise as for example, a patient standing close to an RFID tag-based security system may receive RF energy that activates system componentry. In another example, inductive power may be received by a circuit when the patient stands close to speakers at a concert or is near an MRI system. Thus an optional confirmation step is provided at 804 to prevent inadvertent activation. Block 804 may be omitted in some systems. The system, having been powered up and receiving a stable power input, and having confirmed appropriate activation, then issues therapy as indicated at 806. The system/method described for FIG. 11 may be considered a more or less passive system, in that it only issues therapy when externally powered. In other examples, blocks 800, 802 and 804 may be subsumed in a single step of determining that a scheduled therapy is due, or determining that an external device has made contact, such as through an RF wakeup and handshake protocol that initiates communication, and the external device has commanded therapy delivery. In still other examples, a device may receive and store power passively over a long period of time, and the power on sequence 802 may take place when sufficient power is stored to support a therapy session.

FIG. 12 shows another example. Here, steps performed by an external device are to the left at 900, and steps performed by the implant are to the right at 920. The steps may occur in various sequences.

In an example, an external device issues power at 902 using any of the modes of power delivery described above (radio frequency, inductive, mechanical, optical). That power is received by the implant at 922, which optionally powers on at 924. The implant (also optionally) confirms its activation at 926, as described above to prevent inadvertent activation. The implant then issues therapy at 928. With therapy issuing in the implant at 928, the external device performs a patient check 904. The external may perform step 904 at a specified interval, with the expectation that an implant will begin issuing therapy within a prespecified time period after step 902 takes place. Alternatively, the external may receive a communication from the implant, or may be capable of detecting therapy outputs by the implant, triggering step 904. Step 904 may also be performed repeatedly, in response to detected conditions and/or at intervals, during therapy, before and after therapy, between therapies, etc.

The patient check 904 may be a query that requests the patient to provide one or more indications of whether the therapy is “working,” such as, for example and without limitation, requesting that a patient who is receiving a retinal therapy indicate whether phosphenes are being observed, which would indicate whether the therapy is being delivered in a manner that exceeds the action potential threshold in the affected neurons. A patient check 904 may also be performed using a sensing apparatus, such as by sensing evoked action potentials (whether neural, as in phosphenes, or muscle, such as with a ciliary muscle therapy) with an implanted device or by sensing evoked action potentials with an external device. A patient check 904 may also include determining if therapy is uncomfortable for the patient. For example, if phosphenes are too intense, or the patient is experiencing pain with therapy on, the therapy may be occurring with too much intensity, suggesting reduced amplitude or other power measure should be used. If therapy delivery is not causing a desired response, or if therapy delivery is causing an undesired response, the external device may determine an adjustment to be made 906, which will be communicated to the implant. Once an adjustment instruction is received, the implant adjusts therapy, as indicated at 930. Additional details regarding testing for and using phosphene thresholds are disclosed in US PG Pat. Pub. No. 2020/0101290, the disclosure of which is incorporated herein by reference.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-19. (canceled)

20. An implantable system for treatment of an eye disease comprising:
an implantable device having at least one electrode thereon, a control circuit, and an implantable transducer for receiving a power signal, the implantable device sized and shaped for placement in, on or adjacent to the eye such that issuance of an electric signal with the electrodes has an effect on cells of the eye; and
an external device having an external transducer for issuing a power signal to the implantable transducer;
wherein the control circuit of the implantable device is configured to receive power from the implantable transducer and route electrical signals to the electrodes for treatment of a disease of the eye; and
wherein the power signal generated by the external device is unrelated to any visual appearance of an object.

21. The system of claim 20, wherein the implantable transducer is an antenna adapted to receive an RF signal and convert at least some of the RF signal into electrical power, and the external transducer is an antenna configured to generate an RF signal.

22. The system of claim 20, wherein:
the implantable transducer and external transducer are adapted, respectively, to receive and transmit a light signal;
the light signal has an infrared wavelength; and
the implantable transducer and external transducer are configured to operate using the infrared wavelength.

23. The system of claim 20, wherein the implantable transducer and external transducer are adapted, respectively, to receive and transmit an ultrasound signal.

24. The system of claim 20, wherein:
the implantable transducer is an inductive coil;
the external transducer is an inductive coil;
the external transducer is configured to generate an electromagnetic signal; and
the implantable transducer is configured to receive the electromagnetic signal and generate electrical power from at least a portion thereof.

25. The system of claim 20, wherein the implantable transducer comprises at least first and second transducing units that are separately activatable, and the control circuit is configured to selectively activate the electrodes depending on which transducing unit is activated.

26. The system of claim 20, wherein the signal generated by the external device comprises both data and power, and the implantable device further comprises a demodulation circuit to extract data from the signal generated by the external device.

27. The system of claim 20, wherein:
the eye disease is macular degeneration; and
the implantable device comprises one or more retinal tacks for securing the implantable device to the retina of a patient.

28. The system of claim 27, wherein the implantable device defines an opening surrounded by the electrodes, the opening sized and shaped to avoid blocking light entering the eye from impinging on a majority of the macula when the device is implanted at the retina of the patient.

29. The system of claim 28 wherein the opening has a diameter in the range of about 1.5 to 8 millimeters.

30. The system of claim 20, wherein the disease is presbyopia, and the electrical signals are configured for affecting the ciliary muscle.

31. The system of claim 20, wherein the control circuit is programmable to define an output pattern using the electrodes as anodes and cathodes in a programmable sequence.

32. A method of treating a patient having an eye disease comprising implanting an implantable device in a position on the retina and over or in proximity with the macula such that the fovea centralis is not covered by the implantable device, the implantable device having a plurality of electrodes thereon, wherein the implanting step is performed to place the electrodes in contact with the retina.

33. The method of claim 32 wherein the implantable device comprises a plurality of electrodes that surround an opening, wherein the method of implantation includes placing the opening approximately over the fovea.

34. The method of claim 33 wherein the opening has a diameter in the range of about 1.5 to 8 millimeters.

35. The method of claim 33 further comprising delivering electrical outputs between the electrodes to stimulate neural activity in the maculae and/or fovea.

36. The method of claim 35 wherein the electrical outputs are of sufficient power to cause the patient to experience phosphenes.

37. The method of claim 35, further comprising transmitting power to the implantable device from an external device, and the implantable device using the transmitted power to generate the electrical outputs.

38. A method of treating a disease of the eye comprising:
with an external device, issuing a power signal in the vicinity of or directed into the eye;
with an internal device located inside the eye, receiving the power signal and converting it to an electrical output; and
with the internal device, issuing, through electrodes of the internal device, electrical outputs to retinal cells to stimulate said retinal cells;
wherein the electrical outputs are unrelated to any image or scene.

39. The method of claim 28, wherein the internal device is attached to the retina in the vicinity of but not covering the fovea.
